(19) United States
(12) Patent Application Publication
(10) Pub. No.: US 2014/0179794 A1
(43) Pub. Date: Jun. 26, 2014

(54) LEVOMILNACIPRAN-BASED DRUG FOR FUNCTIONAL RECOVERY AFTER ACUTE NEUROLOGICAL EVENTS

(71) Applicant: Pierre Sokoloff, Belleserre (FR)

(72) Inventor: Pierre Sokoloff, Belleserre (FR)

(73) Assignee: PIERRE FABRE MEDICAMENT, Boulogne-Billancourt (FR)

(21) Appl. No.: 14/235,303

(22) PCT Filed: Jul. 27, 2012
(86) PCT No.: PCT/EP2012/064764

§ 371 Date: Jan. 27, 2014

(30) Foreign Application Priority Data
Jul. 28, 2011 (FR) 1156917

(51) Int. Cl. A61K 31/165

(52) U.S. Cl. CPC A61K 31/165
USPC 514/619

(57) ABSTRACT

The present invention concerns the use of levomilnacipran as medicinal product in functional recovery after a cerebrovascular accident or traumatic brain injury. The pharmaceutical compositions containing levomilnacipran are exclusively those not containing dextromilnacipran to a proportion of more than 5% by weight of the levomilnacipran/dextromilnacipran mixture, to avoid compromising functional recovery due to the alpha1-blocking property of dextromilnacipran.

There are two types of acute neurological events leading to motor and cognitive deficit: one is of vascular origin i.e. a Cerebrovascular Accident (stroke) and the other is of traumatic origin i.e. Traumatic Brain Injury.

According to WHO, a Cerebrovascular Accident (CVA) is “rapidly developing clinical signs of focal (at times global) disturbance of cerebral function, lasting more than 24 hours or leading to death with no apparent cause other than that of vascular origin”. The term brain attack is also used or apoplexy. CVA is to be distinguished from a transient ischemic attack (TIA) defined as “sudden loss of cerebral or ocular function lasting less than 24 hours assumed to be due to an embolism or vascular thrombosis”. CVA is the most frequent type of neurological disease: in Western countries it represents the third cause of death (after coronary diseases and cancers) and the leading cause of handicap acquired at adult age and the second cause of dementia (Murray C J, Lopez A D, Mortality by cause for eight regions of the world: Global Burden of Disease Study, Lancet, 1997; 349:1269-1276).

With CVA the vascular problem concerned is either thrombo-embolic (80% of CVAs) due to interrupted blood supply through obstruction of an artery, or hemorrhagic (20% of CVAs) through rupture of an artery. Cerebral thrombosis is most often caused by arteriosclerosis (hardening and inflammation of the vascular wall). Interrupted circulation secondary to arterial thrombosis (obstruction by a blood clot) is the cause of infarction (death, necrosis of the region concerned) accompanied by softening of the corresponding territory which is no longer irrigated. Gradually the dead tissue is replaced by conjunctive tissue formed of glial cells. Another cause of infarction is a brain embolism in which an atheroma plaque (fat) may detach itself from a large vessel, or when a blood clot is formed for example in embolic cardiopathy (myocardial infarction, valvulopathy, arrhythmia through atrial fibrillation) and comes to obstruct a cerebral artery causing an infarction. Brain hemorrhage may also be due to arteriosclerosis, most often accompanied by arterial hypertension. Brain hemorrhages may also be caused by congenital arterial malformation, infection, brain tumor, or even an upsetting event, an emotion or strenuous effort. Hemorrhage is the origin of hematoma formation which gradually resolves.

CVA diagnosis is firstly clinical. Examination of motor capacities and sensitivity of all or part of the body directs diagnosis towards the site of the lesions which is confirmed by brain imaging. Diagnosis may give rise to problems in comatose, aphasic or amnesic patients. The seriousness of clinical signs varies from the lack of any notable sign to death within a period of a few days and may include motor, coordination and walking disorders, and disorders of sensitivity, speech, visual field, memory and psyche.

Treatment of CVA is started immediately after the event and takes into account the ischemic or hemorrhagic origin, determined by brain imaging using CT brain scans with or without contrast agent, and magnetic resonance imaging (MRI). To treat ischemic CVA the goal is to regulate hydroelectrolytic balance and arterial pressure and to obtain reperfusion of the injured territory using thrombolytic agents such as anti-platelet agents (aspirin) and fibrinolytics (e.g. rt-PA (recombinant tissue plasminogen activator)) when the CVA is taken in charge less than 4 h30 after the first signs. For hemorrhagic CVA, surgery is indicated if it is possible taking into account the topography and volume of the hematoma, the patient's level of consciousness and general condition. Recovery, after the acute phase, is very progressive and may take several months or years. It often requires rehabilitation to treat speech and/or walking disorders. While motor disorders (movements) and sensory disorders (sensation) can generally be restored intellectual sequels may be irreversible.

Traumatic brain injury (TBI) is the main cause of death and severe handicap before the age of 45. The main causes are: road accidents (about 50%), sports accidents, occupational accidents, domestic accidents, attacks, natural disasters and acts of war. There are different types of TBI:
concussion: jarring of the brain subsequent to a violet blow to the brain, whether or not accompanied by initial or temporary loss of consciousness, with no visible radiological lesion to the brain. Return to consciousness spontaneously occurs after a few seconds, minutes or hours after the traumatic event in relation to the extent of the shock and may leave transient memory disorders, even secondary complications: extra-dural hematoma, sub-dural hematoma, cerebral edema.
brain contusion: in this case, there are anatomical lesions of the brain (hemorrhagic necrosis with edema), not necessarily at the site of impact, which may become complicated with brain edema.
immediate deep coma: this is the most serious form of concussion. The patient is in a deep, persistent coma after the shock since the dysfunction of the ascending reticular activating system lies at deeper depth. Signs of decerebration are possible indicating the presence of diffuse mesencephalic and axonal lesions related to concentric propagation and the concentration of the shock waves towards the centre of the brain (stereotaxic phenomena).

The management of TBI includes the search by brain imaging for surgically curable lesions (hematoma), surgery on operable lesions or if not intensive care medical treatment in a specialized unit (anti-edema, pulmonary resuscitation etc.). Diuretics are used to reduce brain edema, and mannitol to dehydrate the brain tissue. At times brain edema is extensive enough to initiate brain herniation (engaging of the lower part of the brain underneath the falx cerebri towards the contralateral cerebral hemisphere, engaging of the lower part of the brain into the foramen magnum). Meningeal hemorrhage may also be associated with brain contusion, translating as headaches, stiff neck and alertness disorders. Clinical and radiological monitoring is set up after emergency treatment. Prognosis depends on the extent of the initial lesions, patient age and general condition before the event. The more the coma is superficial and the younger the patient in good health before the event the greater the chances of recovery. However coma may lead to brain death in some cases.

After the critical period following after the traumatic event and return to consciousness, as is the case with CVA, there is a period of functional recovery which may leave neurological sequels: signs of plegia or paralysis, balance disorders, symbol disorders of aphasia or agnosia type, signs of lesions to the cranial nerves; neuroendocrine disorders: diabetes insipidus, weight loss, fatigue, dizziness, loss of libido and impotency; mental disorders: anxiety after awareness of potentially irreversible sequels, anhedonia. Other consequences are less frequent: subsequent post-traumatic epilepsy, vascular disorders such as aneurysm rupture or brain arterial thrombosis.

The field of the invention concerns medical action with the goal of improving recovery and functional rehabilitation after an acute neurological event whether a CVA or TBI. Within the context of the invention, improving recovery and functional rehabilitation means accelerating and amplifying the resolving of motor, neurophysiological, cognitive or psychiatric symptoms whose onset occurred at the time of the neurological event, or one or more of these symptoms. According to the invention, the motor, neurophysiological, cognitive and psychiatric symptoms include but are not limited to:
paralysis and plegia, including hemiplegia and tetraplegia;
paresthesia or sensitivity disorders;
coordination disorders, ataxia of the limbs and walking;
eye movement disorders;
swallowing disorders;
speech disorders whether concerning perception and understanding or expression;
apraxia and disrupted spatial orientation;
sight disorders and disorders of the visual field;
pupil anomalies;
attention disorders;
memory disorders affecting short-term memory (recent events) or long-term memory (past events;
perception disorders, essentially visual concerning recognition of objects, images writing or physiognomies;
disorders of executory functions such as action planning;
anxiety;
anhedonia and depressive symptoms;
perseverance;
impulsiveness.

The invention also concerns recovery and functional rehabilitation after a recurrent neurological event occurring after an initial neurological event, caused by the consequence thereof on postural balance, loss of sight or visuospatial neglect.

Studies in animal and man have shown that it is possible to accelerate or amplify functional recovery after an acute neurological event, via the administration of medical treatment immediately or even after a period of a few days to a few months following after the event. In animal models of unilateral occlusion of the middle cerebral artery, which mimic CVA, and models of focal cortical lesions, which mimic TBI (Goldstein L B. Basic and clinical studies of pharmacological effects on recovery from brain injury, J. Neural Transplant & Plasticity, 1993, 4:175-192; Feeney D M, de Smet A M, Rai S, Noradrenergic modulation of hemiplegia: facilitation and maintenance of recovery. Restor Neurol & Neurosci, 2004, 22:175-190), medical treatments which proved to be active in the rat and cat are:
amphetamine, a product which increases extracellular levels of noradrenaline, serotonin and dopamine,
transplantation of chromaffin cells, which secrete noradrenaline;
intracerebral infusion of noradrenaline, but not serotonin or dopamine;
administration of a noradrenaline precursor;
administration of alpha-adrenergic agonists.

Clinical studies, in small groups of patients, have shown the possibility of improving motor recovery after CVA by treatment with amphetamine (Sonde L, Nordstrom M, Nilsson C G, Lökk J, Viitanen M, A double-blind placebo-controlled study of the effects of amphetamine and physiotherapy after stroke. Cerebrovasc Dis, 2001,12:253-257); L-DOPS, a noradrenaline precursor (Nishino K, Sasaki T, Takahashi K, Chiba M, Ito T, The norepinephrine precursor L-threo-3,4-dihydroxyphenylserine facilitates motor recovery in chronic stroke patients. J. Clin Neurosci, 2001, 8:547-550), methylphenidate, an agent that also increases the extracellular rates of noradrenaline, serotonin and dopamine (Tardy J, Pariente J, Leger A, Dechamont-Palacin S, Gerdelat A, Guiraud V, Conchou F, Albucher J F, Marque P, Franceries X, Cognard C, Rascol O, Chollet F, Loubinoux I, Methylphenidate modulates cerebral post-stroke reorganization, Neuroimage, 2006, 33: 913-922), reboxetine, a selective inhibitor for the reuptake of noradrenaline (Zitel S, Weiller C, Liepert J, Reboxetine improves motor function in chronic stroke. A pilot study, J. Neurol, 2007, 254:197-201), fluoxetine, a selective inhibitor for the reuptake of serotonin (Pariente J, Loubinoux I, Carel C, Albucher J F, Leger A, Manelfe C, Rascol O, Chollet F, Fluoxetine modulates motor performance and cerebral activation of patients recovering from stroke, Ann Neurol, 2001, 50:718-729). The effect of fluoxetine administered for 3 months was confirmed in a larger double-blind, placebo-controlled clinical study (Chollet F, Tardy J, Albucher J F, Thalamas C, Berard E, Lamy C, Bejot Y, Deltour S, Jaillard A, Niclot P, Guillon B, Moulin T, Marque P, Pariente J, Arnaud C, Loubinoux I, Fluoxetine for motor recovery after acute ischaemic stroke (FLAME): a randomized placebo-controlled trial. Lancet Neurol, 2011, 10:123-30).

It is important to point out that these therapeutic approaches are not intended to restore or protect the injured brain area but to enable the brain which has some plasticity to reorganize its circuits to allow non-injured regions to ensure the functions normally carried out by the injured region, whether motor, neurophysiological or cognitive functions. This was confirmed by functional imagery after CVA and TBI. The ability of the monoamines (noradrenaline, serotonin, dopamine) to promote the functional reorganization of the brain tallies with their known neurotrophic role for developing neurons to ensure the differentiation and survival of neurons.

From these data showing the crucial role of serotonin and noradrenaline in functional recovery, it is concluded that a medicinal product which produces a rise in extracellular levels of both noradrenaline and serotonin would have an advantage over medicinal products which only increase serotonin levels, such as fluoxetine, for treatment after an acute neurological event.

Levomilnacipran is the (1S, 2R) enantiomer of milnacipran (Z(±)-2-aminomethyl)-N,N′-diethyl-1-phenyl cyclopropane carboxamide) described in patents WO 2004/075886 and WO 2009/127737. Milnacipran is an inhibitor of the reuptake of noradrenaline and serotonin having a balanced effect on these two neurotransmitters (Briley M, Prost J F, Moret C, Preclinical pharmacology of milnacipran. Int Clin Psychopharmacol, 1996 Suppl 4:9-14; Preskorn S H, Milnacipran: a dual norepinephrine and serotonin reuptake pump inhibitor, J Psychiatr Pract, 2004, 10:119-26). Milnacipran is a drug used in depression (Spencer C M and Wilde M I, Milnacipran: a review of its use in depression. Drugs, 1998, 56:405-427) and in fibromyalgia (Owen R T, Milnacipran hydrochloride: its efficacy, safety and tolerability profile in fibromyalgia syndrome. Drugs Today (Barc) 2008, 44:653-60). Patent applications WO2003/039598, WO2003/068211, WO2003/077897, WO2003/090743, WO2004/009069, WO2004/030633, WO2004/045718, WO/2007/038620, WO2008/019388, WO2008/021932 and WO2008/147843 also describe the use of milnacipran and its enantiomers in chronic fatigue syndrome, attention deficit with hyperactivity, visceral pain syndromes, functional somatic syndromes, cognitive and sleep disorders, irritable bowel syndrome, chronic lumbar pain, chronic pelvic pain, interstitial cystitis, non-cardiac chest pain, neuropathic pain, temporomandibular joint disorder, atypical facial pain, tension headache, multiple chemical sensitivities, chronic pain associated with medical treatment or radiotherapy or other indications of chronic pain; in particular these patent applications do not describe the use of milnacipran for the treatment of CVA and TBI.

Levomilnacipran is the isomer deemed to be the active isomer of milnacipran; it has the highest affinity for noradrenaline and serotonin transporters compared with that of the other enantiomer, dextromilnacipran, and blocks the reuptake of noradrenaline and serotonin at lower concentrations than those required by dextromilnacipran (Example 1). Surprisingly however dextromilnacipran is the most powerful isomer on the alpha1-adrenergic receptor in rat or man (Example 1). In addition, dextromilnacipran has alpha1-antagonist behavior: it does not activate the recombinant human alpha1 receptor and antagonizes the effect of adrenaline (Example 2).

Preclinical and clinical data indicate that the alpha1-adrenergic receptor plays a crucial role in functional recovery after a neurological event. Indeed a single administration of prazosin, a selective antagonist of the alpha1 receptor (Hoffman and Lefkowitz, Catecholamines, sympathomimetic drugs and adrenergic receptor antagonists, in Goodman and Gilman's, The Pharmacological Basis of Therapeutics, Hardman J G, Limbird L E, Molinoff P B, Ruddon R W publishers, 9^{th} edition, 1995, McGraw-Hill, New York, pp. 229) delays functional recovery after unilateral focal traumatic contusion of the sensorimotor cortex in the rat (Feeney D M and Westerberg V S, Norepinephrine and brain damage: alpha noradrenergic pharmacology alters functional recovery after cortical trauma. Can J Psychology, 1990, 44: 233-252) and precipitates the re-onset of motor symptoms in the rat up to 6 months after a unilateral frontal lesion in the rat when motor functional recovery has been effective (Stibick D L and Fennec D M, Enduring vulnerability to transient reinstatement of hemiplegia by prazosin after traumatic brain injury, J. Neurotrauma, 2001, 18:303-312). In healthy volunteers, the administration of prazosin decreases the efficacy of motor training in inducing cerebral plasticity in the cortex, in the absence of any change in cortical-motor excitability (Sawaki L, Werhahn K J, Barco R, Kopylev L, Cohen L G, Effect of an alpha1-adrenergic blocker on plasticity elicited by motor training, Exp Brain Res, 2003, 148:504-508). Training-induced plasticity is assumed to contribute towards motor functional recovery after an acute neurological event. Goldstein et al (The influence of drugs on the recovery of sensorimotor function after stroke, J Neuro Rehab, 1990, 4:137-144) conducted a study in CVA patients and found that those who were prescribed drugs having deleterious effects on functional recovery in experimental animals, which notably included prazosin, had lower motor scores on the Fugl-Meyer scale than those not given these drugs with deleterious effects, over a 30-day prospective study following after inclusion. All these preclinical and clinical data show that an alpha1-adrenergic antagonist must not be administered to a patient in functional recovery phase after an acute neurological event.

It has therefore surprisingly been discovered that it would be contra-indicated to administer dextromilnacipran, which was found to be an alpha1-adrenergic antagonist when developing the invention, to a patient in functional recovery after an acute neurological event whether a CVA or TBI. Therefore contrary to the provision made in application WO2006/006617, the milnacipran racemate which contains an equal proportion of levomilnacipran and dextromilnacipran, must not be prescribed in the above-mentioned clinical situations. On the contrary, substantially pure levomilnacipran or a levomilnacipran/dextromilnacipran mixture containing dextromilnacipran in a proportion not exceeding 5% by weight of the said mixture (see Example 3) should be used during functional recovery after an acute neurological event whether a CVA or TBI.

Patent WO2004/075886 claims the use of levomilnacipran to prepare a medication for a variety of pathologies in patients presenting with cardiovascular risk, on the basis of the observation that levomilnacipran induces fewer hemodynamic phenomena than the milnacipran racemate in dogs. However, this patent does not disclose the particular activity of dextromilnacipran on the alpha1-adrenergic receptor and even less so the use of levomilnacipran for functional recovery after CVA or TBI.

Further, according to the invention, levomilnacipran is used in the form of a pharmaceutically acceptable salt chosen from among the inorganic acid addition salts non-toxic for patients in whom they are administered. The term “pharmaceutically acceptable” refers to molecular entities and compositions which do not produce any adverse or allergic effect or other undesirable reaction when administered to man or animal. Examples of pharmaceutically acceptable acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate salts and the like (see for example Berge S M, Bighley L D, Monkhouse D C, Pharmaceutical salts, 1977, 66:1-19). The preferred salt however in the present invention is levomilnacipran hydrochloride.

The invention also concerns a pharmaceutical composition characterized in that it contains levomilnacipran as active ingredient and at least one pharmaceutically acceptable excipient. When used herein, the term pharmaceutically acceptable excipient includes any diluent, adjuvant or excipient such as preserving agents, filler agents, disintegrating, wetting, emulsifying, dispersing, antibacterial or antifungal agents, or agents which can delay intestinal and digestive absorption and resorption. The use of these media or vectors is well known to the person skilled in the art.

The pharmaceutical compositions may contain substantially pure levomilnacipran or mixtures of levomilnacipran and dextromilnacipran, provided that the proportion of dextromilnacipran is insufficient for the alpha1-adrenergic antagonist activity to be significant and for the patient to be exposed to blocking of the alpha1-adrenergic receptor. A simulation of the activity of the levomilnacipran/dextromilnacipran mixtures, confirmed by experimental data, shows that the anti-alpha1 activity becomes significant with mixtures containing more than 5% of dextromilnacipran (Example 3). The proportion of dextromilnacipran in a levomilnacipran/dextromilnacipran mixture must not therefore exceed 5% by weight of the said mixture.

The pharmaceutical compositions according to the present invention can be formulated for administration to mammals, including man. The compositions of the invention can be administered via oral, sublingual, sub-cutaneous, intramuscular, intravenous, transdermal, local or rectal route. In this case, the active ingredient can be administered in unit administration forms, in a mixture with conventional pharmaceutical carriers, to animal or human beings. The suitable unit administration forms comprise the forms via oral route such as tablets, capsules, powders, granules, each containing a predetermined quantity of levomilnacipran, they also include oral solutions or suspensions in an aqueous liquid or non-aqueous liquid, or an oil/water or water/oil liquid emulsion, sublingual and mouth administration forms, sub-cutaneous or transdermal, topical, intramuscular, intravenous, intra-nasal or intraocular administration forms and rectal administration forms. When a solid composition is prepared in tablet form, the levomilnacipran is mixed with a pharmaceutical vehicle such as gelatin, starch, lactose, magnesium stearate, talc, gum Arabica, silica or the like. It is possible to coat the tablets with sucrose or other suitable materials.

The release of the said active ingredient can be delayed to obtain sustained release so as to allow the administration of a single daily dose. Said galenic formulation can be obtained following the method described in patent EP 939 626 or any other method.

A capsule preparation is obtained by mixing the active ingredient with a diluent and pouring the mixture obtained into soft or hard capsules.

A preparation in syrup or elixir form can contain the active ingredient combined with a sweetener, an antiseptic, and a suitable flavoring agent and coloring agent.

Powders or water-dispersible granules can contain the active ingredient in a mixture with dispersing or wetting agents, or suspending agents, and also with flavor enhancing or sweetening agents.

For rectal administration, recourse is had to suppositories prepared with binders melting at rectal temperature e.g. cocoa butter or polyethylene glycols.

For parenteral administration (intravenous, intramuscular, intradermal, sub-cutaneous), intra-nasal or intra-ocular administration, aqueous suspensions are used, isotonic saline solutions or sterile solutions for injection which contain pharmaceutically compatible dispersing and/or wetting agents.

The active ingredient may also be formulated in the form of microcapsules optionally with one or more additive carriers.

Advantageously the pharmaceutical composition according to the present invention is intended for administration via oral route.

The dosages of the pharmaceutical compositions containing levomilnacipran in the compositions of the invention are adjusted to obtain a quantity of active substance which is efficient to obtain the desired therapeutic response for a composition particular to the administration route. The chosen dosage level therefore depends on the desired therapeutic effect, the chosen route of administration, the desired length of treatment, the weight, age and gender of the patient, the sensitivity of the individual to be treated. As a result, the optimal dosage must be determined in relation to parameters deemed to be relevant by specialists in the field.

Preferably the levomilnacipran is administered in pharmaceutically acceptable compositions in which the daily dose of levomilnacipran, expressed as base amount, is between 25 and 200 mg taken in a single administration or several times per day. Further preferably, the pharmaceutical composition allows modified intestinal absorption so that a single administration per day is sufficient.

### EXAMPLE 1

Measurement of the affinity of the two isomers of milnacipran for the noradrenaline and serotonin transporters and for the alpha1-adrenergic receptor.

The affinities of levomilnacipran and dextromilnacipran were measured on the binding to the recombinant human transporters of noradrenaline and serotonin, and on the binding to the human recombinant alpha1 receptor. The inhibition by these two products of the reuptake of noradrenaline [³H] and serotonin [³H] was also measured.

Methods:
Binding to the noradrenaline transporter: binding was measured on membranes of MDCK cells expressing this transporter, purchased from Perkin-Elmer (batch N^{o} 418-165-A), diluted in 50 mM TRIS-HCl buffer containing 120 mM NaCL and 5 mM KCl at a concentration of 5 μg proteins, in the presence of 2 mM N-methyl-nisoxetine [³H] and increasing concentrations of levomilnacipran or dextromilnacipran (10⁻¹¹ to 10⁻⁵ M). The bound fraction was separated by filtration and washing in cooled TRIS+NaCl+KCl buffer. Non-specific binding was measured in the presence of 10 μM desipramine.
Binding to the serotonin transporter: binding was measured on membranes of MDCK cells expressing this transporter, purchased from Perkin-Elmer (Batch N^{o} 316-199-A) diluted in 50 mM TRIS-HCl buffer containing 120 mM NaCl and 5 mM KCl at a concentration of 5 μg of proteins, in the presence of 2 nM citalopram [³H] and increasing concentrations of levomilnacipran or dextromilnacipran (10⁻¹¹ to 10⁻⁵ M). The bound fraction was separated by filtration and washing in cooled TRIS+NaCl+KCl buffer. Non-specific binding was measured in the presence of 10 μM fluoxetine.
Binding to the recombinant human alpha1 receptor: binding was measured on membranes of CHO cells (Chinese Hamster Ovary) expressing the human alpha1B receptor (Wurch T, Boutet-Robinet E A, Palmier C, Colpaert F C, Pauwels P J, Constitutive coupling of chimeric dopamine D2/alpha1B receptor to the phospholipase C pathway: inverse agonism to silent antagonism of neuroleptic drugs, J Pharmacol Exp Ther, 2003, 304:380-390) diluted in 50 mM TRIS-HCl buffer at a concentration of 7.8 μg of proteins, in the presence of 0.1 nM prazosin [³H] and increasing concentrations of levomilnacipran or dextromilnacipran (10⁻¹¹ to 10⁻⁵ M). The bound fraction was separated by filtration and washing in cooled TRIS buffer. Non-specific binding was measured in the presence of 10 μM phentolamine.
Reuptake of noradrenaline [³H]: CHO-K1 cells were permanently transfected with the gene of the human transporter of noradrenaline by electric pulsing (Biorad gene pulser) and the transfected clones were then selected by incubation in geneticin. To measure reuptake, the transfected cells were cultured in 24-well plates then incubated in the presence of pargyline and ascorbate (100 μM) and noradrenaline [³H] (specific activity=40.7 Ci/mole) at a concentration of 10 nM. Reuptake was halted by aspiration and rinsing the medium and the radioactivity captured by the cells was counted by liquid scintillation. The non-specific signal was determined in the presence of 10 μM desipramine.
Reuptake of serotonin [³H]: CHO-K1 cells were transfected permanently with the gene of the human transporter of serotonin by electric pulsing (Biorad gene pulser) and the transfected clones were selected by incubation in geneticin. To measure reuptake, the transfected cells were cultured in 24-well plates then incubated in the presence of pargyline and ascorbate (100 μM) and serotonin[³H] (specific activity=32.7 Ci/mole) at a concentration of 10 nM. Reuptake was halted by aspiration and rinsing of the medium and the captured radioactivity by the cells was counted by liquid scintillation. The non-specific signal was determined in the presence of 10 μM fluoxetine.

### Results:

Table 1 gives the values of inhibition constants (Kᵢ) of levomilnacipran and dextromilnacipran for the serotonin and noradrenaline transporters, and of the alpha1-adrenergic receptor.

TABLE 1

| | Value of Kᵢ for binding or of IC₅₀ | |
|---|---|---|
| | for reuptake (μM) | |
| Target | levomilnacipran | dextromilnacipran |
| Binding to the | 0.091 | 10.5 |
| noradrenaline | | |
| transporter (NET) | | |
| Binding to the | 0.011 | 0.32 |
| serotonin | | |
| transporter | | |
| (SERT) | | |
| Inhibition of | 0.010 | 0.15 |
| noradrenaline | | |
| reuptake [³H] | | |
| Inhibition of | 0.018 | 0.28 |
| serotonin | | |
| reuptake [³H] | | |
| α1A adrenergic | 110 | 3.4 |
| receptor | | |

### EXAMPLE 2

Measurement of the intrinsic activity of dextromilnacipran on recombinant human alpha1A and alpha1B receptors.

The intrinsic functional activity of dextromilnacipran was measured on cells expressing the human alpha1A and alpha1B receptors to determine the agonist/antagonist property thereof.

Methods:

CHO-K1 cells having stable expression of the human alpha1A receptor or human alpha1B receptor were obtained using the described method (Vicentic et al. Biochemistry and pharmacology of epitope-tagged alpha1a-adrenergic receptor subtype, J Pharm Exp Ther, 2002, 302-58-65). The agonist activity was evaluated by fluorimetry measurement of the intracellular concentration of calcium following a conventional technique using a fluorescent calcium chelator and signal recording with a Fluorometric Imaging Plate Reader (FLIPR, Molecular Devices, Saint-Grégoire, France). As positive reference (−)adrenaline was used, and responses were then normalized to the response of (−)adrenaline at a concentration of 10 μM.

Results:

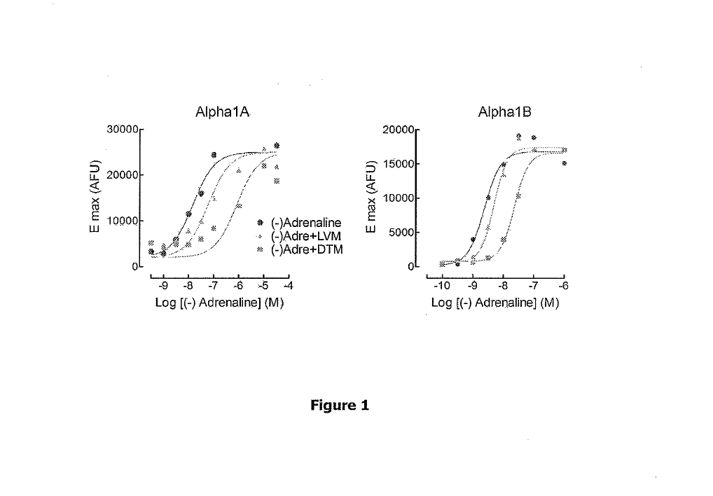

Over a concentration range of 3.10⁻⁷ M to 10⁻³ M, dextromilnacipran did not show any agonist activity higher than 10% of the activity of the (−)adrenaline, whether on the alpha1A receptor or alpha1B receptor. The (−)adrenaline was then incubated in increasing concentrations (3.10⁻¹⁰ to 3.10⁻⁵ M) in the presence of levomilnacipran or dextromilnacipran at a concentration of 300 μM. FIG. 1 shows that the concentration-response curve of the (−)adrenaline is shifted towards the right by dextromilnacipran by a factor of about 100 for the alpha1A sub-type and by about 10 for the alpha1B sub-type. For levomilnacipran, the shift is only about 3 for the alpha1A sub-type and 2 for the alpha1B sub-type. It is concluded that dextromilnacipran is an antagonist for these two sub-types of alpha1-adrenergic receptors. Levomilnacipran is also an antagonist of the alpha1A and alpha1B receptors but to a much less powerful extent than dextromilnacipran.

### EXAMPLE 3

Pharmacological Characteristics of Mixtures of Levomilnacipran and Dextromilnacipran, with Varying Proportions of the Enantiomers.

Objectives

Levomilnacipran (enantiomer, 1S, 2R) has a distinct pharmacological profile compared with racemic milnacipran (2207) and the other 1R, 2S enantiomer (dextromilnacipran). Levomilnacipran is the most active enantiomer on the desired targets: binding with the noradrenaline transporter (NET), binding with the serotonin transporter (SERT) and the PCP site (NMDA receptor, glutamate system), but is the least active on non-desired targets i.e. on α1A and α1B adrenergic receptors. We estimated the pharmacological properties of different mixtures (containing varied percentages of dextromilnacipran). First, binding assays were simulated and the apparent inhibition constants of the mixtures for NET, SERT and the α1A receptors were calculated. Next, the simulation was experimentally validated for the α1A receptors for which the variations in levomilnacipran had the most impact.

Methods

Simulation

The inhibition constants (value of KJ of levomilnacipran and dextromilnacipran on the main targets were experimentally determined using conventional binding assays with specific radioligands and recombinant human proteins (see Example 1).

For each target, the total of bound radioligand was calculated taking into account the inhibitor effect of each inhibitor. The total of each occupied target is described using the conventional law of mass action:

$$\begin{matrix}\frac{R . S .}{B} = {K}_{d} & ( 1 ) \\ \frac{R . {I}_{1}}{{RI}_{1}} = {K}_{i 1} & ( 2 ) \\ \frac{R . {I}_{2}}{{RI}_{2}} = {K}_{i 2} & ( 3 ) \\ Bmax = B + {RI}_{1} + {RI}_{2} + R & ( 4 )\end{matrix}$$

where R is the concentration of free binding sites; Bmax is the total concentration of sites; B is the concentration of sites bound to the radioligand; S is the radioligand concentration; K_{d} is the dissociation constant of the radioligand; i₁ is the concentration of inhibitor 1; Kᵢ₁ is the inhibition constant of inhibitor 1; RI₁ is the concentration of sites occupied by inhibitor 1; i₂ is the concentration of inhibitor 2; Kᵢ₂ is the inhibition constant of inhibitor 2 and RI₂ is the concentration of sites occupied by inhibitor 2.

On the basis of equations (1) to (4):

$$B = \frac{Bmax . S}{S + {K}_{d} ( 1 + {i}_{1} / {K}_{i 1} + {I}_{2} / {K}_{i 2} )}$$

The values of B were calculated taking into account the real values of Kᵢ₁ and Kᵢ₂ (see Table above) causing the proportion of dextromilnacipran to vary by 0.01% to 30% in the mixture, and varying the concentration of the mixture from 0.01 to 52.0 μM. The values of K_{d} and S which were used were similar to the values of the binding assays. A theoretical inhibition curve was therefore plotted with each combination of values of the different parameters. Then the apparent IC₅₀ value for each curve was calculated by non-linear regression as per the logistic equation:

Y=100/1+10(i−Log IC₅₀)

where i is the concentration of the inhibitor (mixture) and the apparent value of Kᵢ was derived using the Cheng-Prussoff equation: IC₅₀=Kᵢ (1+S/K_{d}).

### 2.2 Binding Assays

A series of levomilnacipran and dextromilnacipran mixtures with varying proportions of dextromilnacipran was prepared, and each mixture was incubated at varying concentrations with membranes of cells expressing the recombinant human α1A receptor and using prazosin [³H] as radioligand. The apparent IC_{H} value for each curve was calculated by non-linear regression as per the logistic equation:

Y=100/1+10(i−Log IC₅₀)

where i is the concentration of the inhibitor (mixture) and the apparent value of Kᵢ was derived using the Cheng-Prussoff equation: IC₅₀=Kᵢ (1+S/K_{d}).

### Results

The results are expressed as apparent Kᵢ value as a function of dextromilnacipran percentage (FIG. 1).

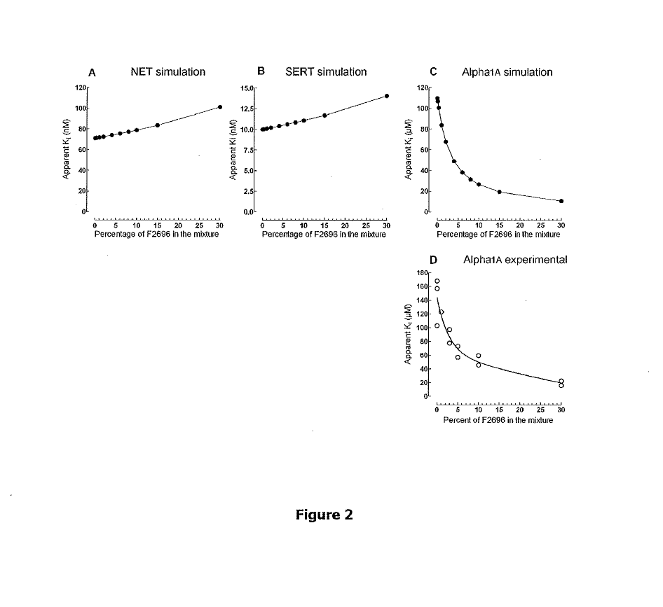

FIG. 2 shows: the apparent Kᵢ values of simulated assays (A, B and C) and measured values (D) of mixtures of levomilnacipran (F2695) and dextromilnacipran (F2696) with increasing proportions of dextromilnacipran for NET, SERT or α1A receptor targets.

The values of Kᵢ for NET and SERT were not too affected by different proportions of dextromilnacipran. On the contrary, the apparent value of Kᵢ for the α1A receptors, whether for simulation assays or for real assays, was dramatically affected when the percentage of dextromilnacipran was increased, which indicates that the impact on the α1 receptors is non-negligible. If it is considered that the impact becomes non-negligible when the value of Kᵢ drops by half, the maximum percentage of dextromilnacipran is about 5%.

### CONCLUSION

A mixture with a proportion of dextromilnacipran higher than this 5% may not be bioequivalent to “substantially pure” levomilnacipran, or to a mixture with smaller proportions of dextromilnacipran. The impact on the α1A receptors of mixtures with proportions of dextromilnacipran higher than 5% is not negligible and such mixtures should not be used in the treatment of functional recovery after a stroke.

1. A levomilnacipran/dextromilnacipran mixture containing dextromilnacipran in a proportion not exceeding 5% by weight of the said mixture for use thereof as medicinal product for recovery and functional rehabilitation after an acute neurological event and recurrences thereof.

2. The mixture according to claim 1 for use thereof in patients diagnosed with cerebral vascular accident of ischemic or hemorrhagic origin.

3. The mixture according to claim 1 for use thereof in patients diagnosed with traumatic brain injury.

4. Pharmaceutical compositions comprising at least one pharmaceutically acceptable excipient and a levomilnacipran/dextromilnacipran mixture containing dextromilnacipran in a proportion not exceeding 5% by weight of the said mixture as active ingredient for use thereof as medicinal product for recovery and functional rehabilitation after an acute neurological event and recurrences thereof.

5. Pharmaceutical compositions according to claim 4 in patients diagnosed with cerebrovascular accident.

6. Pharmaceutical compositions according to claim 4 in patients diagnosed with traumatic brain injury.

7. Pharmaceutical compositions according to any of claims 4 to 6 characterized in that the daily dosage of levomilnacipran is between 50 and 200 mg.

8. Pharmaceutical compositions according to one of claims 4 to 7 characterized in that they are in a modified intestinal absorption form allowing the administration of a single dose per day.
